(19) United States
(12) Patent Application Publication
(10) Pub. No.: US 2025/0268549 A1
(43) Pub. Date: Aug. 28, 2025

(54) Systems, Methods, and Apparatuses for Learning Anatomical Consistency, Sub-Volume Spatial Relationships and Fine-Grained Appearance for Computed Tomography

(71) Applicant: Arizona Board of Regents on behalf of Arizona State University, Scottsdale, AZ (US)

(72) Inventors: Jiaxuan PANG, Tempe, AZ (US); DongAo MA, Tempe, AZ (US); Jianming LIANG, Scottsdale, AZ (US)

(73) Assignee: Arizona Board of Regents on behalf of Arizona State University, Scottsdale, AZ (US)

(21) Appl. No.: 19/059,165

(22) Filed: Feb. 20, 2025

Related U.S. Application Data
(60) Provisional application No. 63/557,408, filed on Feb. 23, 2024.

(51) Int. Cl. A61B 6/00; A61B 6/03; G06V 10/42; G06V 10/771; G16H 30/20

(52) U.S. Cl. CPC A61B 6/5223; A61B 6/032; G06V 10/42; G06V 10/771; G16H 30/20

(57) ABSTRACT

A self-supervised learning framework learns fine-grained features, high-level global features, and contextual relationship features of anatomical structures in medical images of a plurality of patients. The framework receives computed tomography three-dimensional volumes for a number of patients (“patient volumes”) and learns sub-volume relationships within the patient volumes through 3D sub-volume order prediction of correct positions in shuffled sub-volumes. The framework further learns fine-grained image features within the patient volumes through volume appearance recovery from a set of misplaced sub-volumes. Finally, the framework learns high-level global image features of anatomical structures in the patient volumes by maximizing an agreement between two spatially related views through a student-teacher network of the self-supervised learning framework.

### CLAIM OF PRIORITY

This application claims the benefit of U.S. Provisional Patent Application No. 63/557,408, filed Feb. 23, 2024, entitled “SYSTEMS, METHODS, AND APPARATUSES FOR LEARNING ANATOMICAL CONSISTENCY, SUB-VOLUME SPATIAL RELATIONSHIPS AND FINE-GRAINED APPEARANCE FOR COMPUTED TOMOGRAPHY”, the disclosure of which is incorporated by reference herein in its entirety.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments of the invention relate to systems, methods, and apparatuses for implementing a self-supervised learning framework to learn fine-grained features, high-level global features, and contextual relationship features of anatomical structures in medical images of patients.

### BACKGROUND

### 1. Introduction

Deep learning has showcased remarkable success across various visual tasks and is increasingly recognized as a pivotal method in the realm of medical imaging analysis. Obtaining meaningful features from medical images is a critical step in influencing the accuracy of diagnostics and treatment planning. Supervised learning heavily relies on the quantity and quality of annotated data, but annotating medical images is tedious, time-consuming and demands specialty-oriented expertise, especially for segmentation tasks. Segmenting images requires a nuanced understanding of global features, complex anatomical structures, subtle patterns, and variations. Recently, self-supervised learning has emerged as a transformative paradigm. Unlike supervised learning, which relies on labeled datasets for training, self-supervised learning allows models to autonomously generate labels from the data itself. This autonomy is particularly advantageous in scenarios where obtaining large labeled datasets is challenging or impractical, particularly in the medical image analysis domain, in which the necessity for robust, efficient, and effective feature extraction becomes even more pronounced.

FIG. 1 shows four patient Computed Tomography (CT) volumes 100, 105, 110 and 115 across three views, axial view 120, coronal view 125, and sagittal view 130. While various patients share similar anatomical structures, noticeable differences in appearance persist among individuals. Major organs are not only evident in substantial regions but also in smaller areas. What is needed is a model that encompasses not only global anatomical information but also sub-volume relationships and fine-grained appearance information.

As depicted in FIG. 1, substantial similarities are evident in the axial, coronal, and sagittal views 120, 125 and 130 across diverse patients. A robust model according to embodiments of the invention can grasp the overarching concept of shared appearances and features among all patient volumes. Furthermore, leveraging symmetry and the recurring nature of body structures, the model is expected to effectively identify high-level anatomical structures and intra-volume spatial relationships. However, despite the significant similarities observed across all patient volumes, there are still subtle differences present in each individual. This necessitates the model to possess the capability to capture finer-grained features in order to discern and account for patient-level distinctions.

Within the domain of organ segmentation tasks, as illustrated in FIG. 1, substantial regions of interest are evident across all three views, encompassing major organs like the liver and spleen. Additionally, smaller areas of interest, such as the esophagus and adrenal glands, are of significance. Hence, an effective model should not only be capable of capturing organ-specific relationships and appearance features derived from anatomical structures but also be equipped to deliver fine-grained features, for example, at the pixel level, ensuring precise details of smaller regions. To this end, a question is raised: how can one formulate a self-supervised learning proxy task that enables the model to learn fine-grained features, high-level global features, and contextual relationship features? A new self-supervised learning method, according to embodiments of the invention, referred to herein as ASA (learning Anatomical consistency, sub-volume Spatial relationships, and fine-grained Appearance), answers this question. ASA is equipped with three learning perspectives: a) understanding sub-volume relationships through 3D sub-volume order prediction, b) capturing fine-grained features within volumes through volume appearance recovery, and c) acquiring high-level global features by maximizing the agreement between two spatially related views through the student-teacher network, resulting in a robust, effective and efficient pretrained model. The embodiments and related experiments provide the following contributions:

A novel vision transformer-based self-supervised learning framework for 3D medical images that simultaneously captures high-level anatomical information, intra-volume relationships, and fine-grained appearance features.

Introduction of a cyclic pretraining strategy involving a student-teacher network to facilitate learning from multiple perspectives.

Comprehensive experiments showcasing the transferability of ASA across diverse single-organ and multi-organ segmentation tasks, surpassing the performance of multiple fully supervised and self-supervised methods.

An efficient pretrained model that encapsulates rich semantic information, enhancing its utilization efficiency.

Section 2 below provides a brief overview of prior works, emphasizing the innovations introduced by ASA. Section 3 describes the pretraining strategy of the training framework according to embodiments. The pretraining and finetuning protocols are elaborated in Section 4, followed by Section 5, which provides a thorough evaluation of the model and baseline models across various single-organ and multi-organ segmentation tasks, along with an assessment of data efficiency.

### 2. Related Work

Self-supervised learning in computer vision is an approach where a model learns from the data itself without relying on expert annotations. This paradigm leverages inherent structures or relationships within the data to generate supervisory signals for training. Based on the primary learning perspectives, the model may be delineated into the following types: a) rotation prediction, b) distorted image recovery, c) contrastive learning, and d) image context learning, each of which is discussed below.

Rotation prediction. The concept of image rotation was first introduced in Spyros Gidaris, Praveer Singh, and Nikos Komodakis, “Unsupervised Representation Learning by Predicting Image Rotations,” arXiv preprint arXiv:1803.07728, 2018, which is a self-supervised learning task where a model is trained to predict the rotation angle applied to an image. It learns the high-level concept of the object displayed in the image, such as location, type, and organization. In contrast, ASA goes beyond learning solely the high-level image concept by maximizing agreement between two spatial-related views. It also acquires knowledge about the intrinsic anatomical structure within the input sample through order prediction and appearance recovery. This results in a more resilient and efficient model.

Distorted image recovery. This concept refers to a process of reconstructing or restoring an image that has undergone some form of distortion or degradation back to its original, undistorted content. This underlying idea has been widely incorporated as a proxy task into various self-supervised learning works. The distortions can arise from various sources, such as noise, blurring, compression artifacts, or other forms of corruption. The task aims to learn the underlying patterns, structures, and fine-grained features embedded in the image. While ASA shares similarities with these methods in terms of volume appearance reconstruction, ASA sets itself apart by (1) maximizing agreement between two spatial-related views to learn global image features, (2) reconstructing the correct volume from a set of misplaced sub-volumes to grasp fine-grained volume appearances and the underlying structures, and (3) predicting the correct positions of shuffled sub-volumes to capture sub-volume wise contextual features.

Contrastive learning. The contrastive learning has been verified as a promising pretraining method on visual representation learning when transferring to downstream tasks. It aims to differentiate between similar and dissimilar pairs of data. The primary goal of contrastive learning is to learn a useful global and discriminative representation where semantically similar samples are mapped close to each other, while dissimilar samples are pushed apart. In contrast, due to the great similarity shared among medical images, similar learning perspectives are accomplished by maximizing the global consistency between two spatial-related views to learn general volume features.

Image context learning. This learning strategy aims to comprehend and leverage the contextual information within an image. This involves the model learning to recognize patterns, relationships, and spatial dependencies among pixels, regions, or objects within the image. Various pretext tasks have been devised to predict the context arrangement of image patches, including predicting the relative position of two image patches, solving Jigsaw puzzles, playing Rubik's cube, and patch de-shuffling and recovery. Doersch et al., Noroozi and Favaro, Zhuang et al. utilize multi-Siamese CNN backbones as feature extractors, incorporating additional feature aggregation layers to establish relationships between input patches. However, these feature aggregation layers are discarded after the pretraining phase, retaining narrowed features when transferring to target tasks. As a consequence, the relationships learned among image patches are lost in the target tasks. In contrast to these methodologies, the embodiments described herein elaborate the Transformer architecture to capture relationships among anatomical patterns embedded in image patches, ensuring full transferability to target tasks. Furthermore, although the embodiments share similarities with Jiaxuan Pang, Fatemeh Haghighi, DongAo Ma, Nahid Ul Islam, Mohammad Reza Hosseinzadeh Taher, Michael B Gotway, and Jianming Liang, “POPAR: Patch Order Prediction and Appearance Recovery for Self-supervised Medical Image Analysis,” MICCAI Workshop on Domain Adaptation and Representation Transfer, pages 77-87, Springer, 2022, the embodiments differentiate themselves by (1) incorporating a student-teacher network to maximize global consistency between two spatial-related views, facilitating the learning of general volume features, and (2) utilizing a 3-D coordinate representation for sub-volume order prediction to more effectively capture their spatial relationships.

Student-teacher networks. Originating from the field of knowledge distillation, this paradigm involves training a student model to emulate the knowledge encoded in a more complex teacher model. The primary motivation behind this approach is to enhance the generalization and efficiency of the student model by leveraging the rich knowledge contained in the teacher model. In contrast, ASA employs the student-teacher learning paradigm to amalgamate knowledge acquired by students across diverse tasks and learning perspectives into the teacher network. This results in a robust, effective, and efficient pretrained model applicable to downstream tasks.

Swin UNETR. Swin UNETR is a deep-learning architecture designed for semantic segmentation tasks, particularly in medical imaging. It combines the strengths of two prominent models: Swin Transformer and UNETR. The Swin Transformer is known for its effectiveness in capturing long-range dependencies among multiple image scales, while the UNETR is a variation of the classic UNet architecture that incorporates transformer blocks for image segmentation. Swin UNETR leverages a hierarchical set of transformer blocks to capture both local and global context, making it well-suited for tasks requiring a nuanced understanding of spatial relationships in images. Swin UNETR is officially pretrained on three common self-supervised learning tasks, (1) masked volume inpainting to learn the appearance and semantics of visual structures, (2) image rotation to learn angle invariance, and (3) contrastive coding to strengthen intra-class compactness and inter-class separability. As later described herein, the ASA model according to the embodiments yields more effective and efficient features than the Swin UNETR pretrained model.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 depicts four patient volumes across three views.

FIGS. 2A and 2B depict a synthetic illustration of sub-volume order distortion and spatial-related cropping.

FIG. 3 depicts a framework that encompasses two learning perspectives: (1) acquiring high-level global features by optimizing the agreement between two spatially related views through the student-teacher learning paradigm (upper path), and (2) acquiring sub-volume relationships and fine-grained features through sub-volume order prediction and volume appearance recovery (lower path), according to embodiments of the invention.

FIG. 4 assesses the data efficiency of the described embodiments, Swin UNETR, and the Universal Model on the AMOS22 validation dataset.

FIG. 5 provides Table 1 containing experiment results on the BTCV validation dataset to reveal embodiments that surpass both fully-supervised and self-supervised techniques in achieving the highest average Dice score across all organs.

FIG. 6 presents Table 2 containing results of experimentation on single-organ segmentation tasks which demonstrate that embodiments of the invention outperform both state-of-the-art self-supervised learning methods.

FIGS. 7A and 7B provide Table 3, which shows validation of the effectiveness of embodiments using the AMOS2022 dataset, conducting a comparative analysis that contrast the ASA model against Swin UNETR and the Universal Model.

FIGS. 8A, 8B and 8C illustrate circles to depict spatially related embeddings and cross signs for the diagonal embeddings for the Universal Model (FIG. 8A), the Swin UNETR model (FIG. 8B), and the ASA model (FIG. 8C) demonstrating strong relational properties among the spatially related sub-volumes, where circles are closely clustered.

FIG. 9 provides an algorithm for one round of pretraining initiated by embodiments of the invention.

FIG. 10 provides Table 4, showing that a model according to the embodiments that is trained by volume appearance recovery or global consistency tasks that yields comparable performance.

FIG. 11 shows results in Table 5 that indicate when making predictions based on 3D sub-volume order presentation, performance across all downstream tasks consistently surpasses that achieved with 1D sub-volume order prediction, which underscores the importance of utilizing 3D sub-volume order presentation for enhanced model performance.

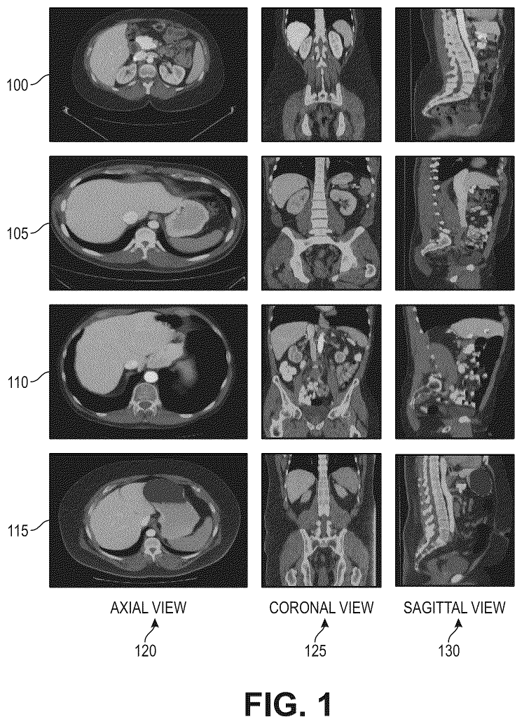

### DETAILED DESCRIPTION

Described herein are systems, methods, and apparatuses for implementing a self-supervised learning framework to learn fine-grained features, high-level global features, and contextual relationship features of anatomical structures in medical images of a plurality of patients.

There is a strong demand for powerful deep-learning approaches to extract robust features from medical images. Supervised learning heavily relies on the quantity and quality of annotated data, but annotating medical images is tedious, laborious, and time-consuming, requiring specialized expertise, especially for segmentation tasks. Segmenting medical images requires not only macroscopic anatomical patterns but also microscopic textural details. Given the intriguing symmetry and recurrent patterns inherent in medical images, the exploration of high-level context, spatial relationships in anatomy, and fine-grained features from anatomical structures in a self-supervised manner can lead to the development of potent models. To this end, embodiments of the invention provide a novel self-supervised learning approach referred to herein as ASA, which is designed to learn anatomical consistency, sub-volume spatial relationships, and fine-grained appearance for 3D computed tomography (CT) images. The novelty of ASA at least in part stems from its utilization of intrinsic properties of medical images, with a specific focus on computed tomography volumes. ASA enhances the model's learning capabilities, encompassing high-level global features, sub-volume relationships, and intricate appearance features. Extensive experimental results described herein validate the robustness, effectiveness, and efficiency of the pretrained ASA model.

### 3. Embodiments

ASA is designed to concurrently acquire anatomical knowledge of (1) intra-volume spatial relationships by sub-volume order prediction and (2) volume-wise fine-grained features by volume appearance recovery. Moreover, it employs the student-teacher learning paradigm to (3) automatically capture high-level features by optimizing the agreement between features from two spatially related crops.

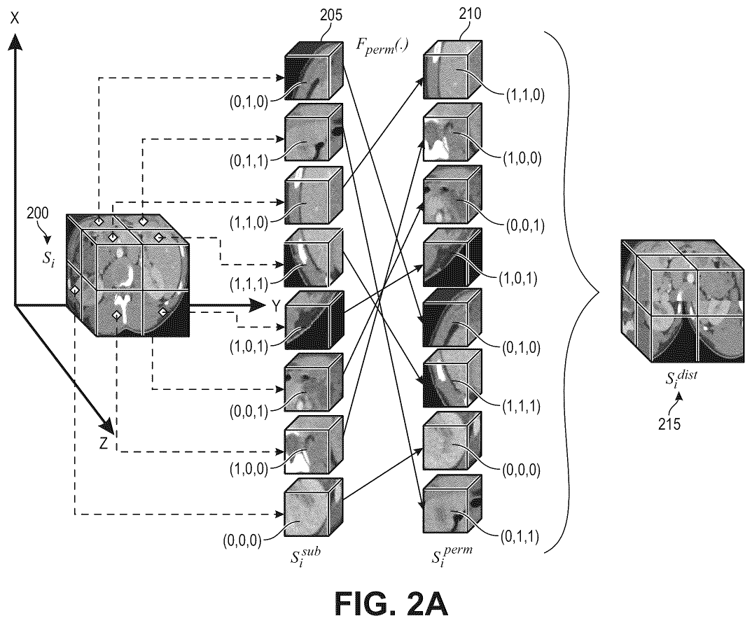

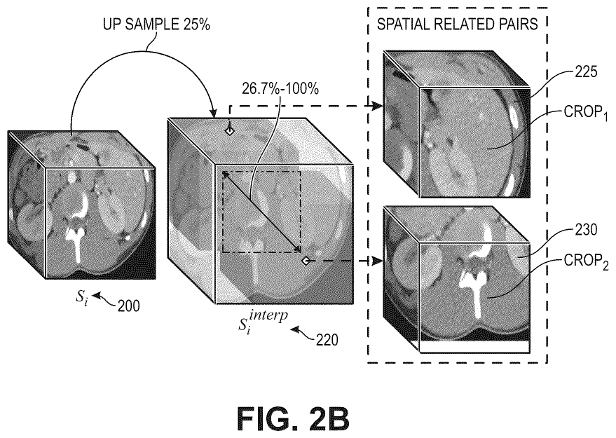

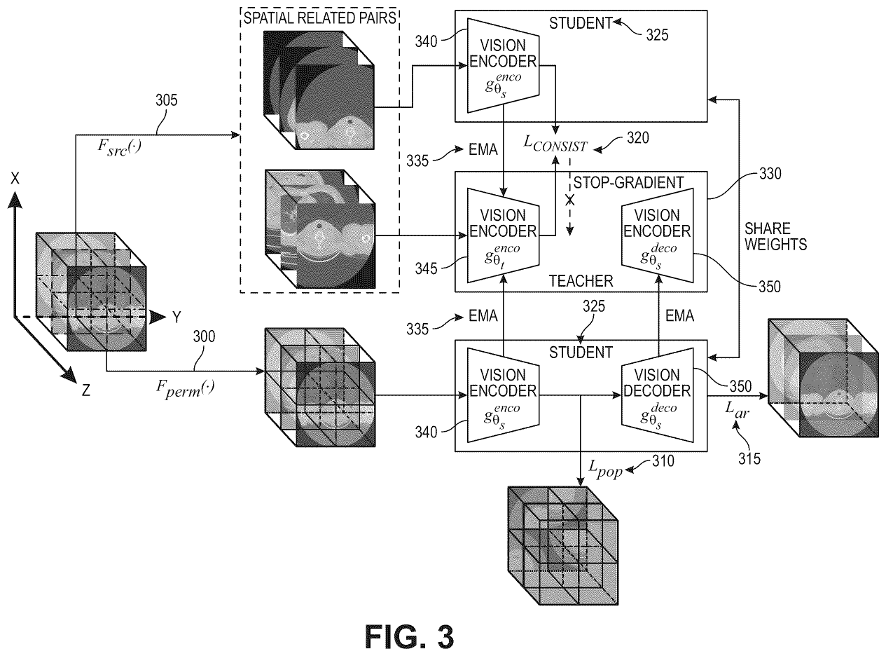

FIGS. 2A and 2B provide a synthetic illustration of sub-volume order distortion and spatial-related cropping. A method according to embodiments of the invention utilizes as depicted in FIG. 2A sub-volume order distortion to simultaneously acquire sub-volume relationships and fine-grained features. The method employs as depicted in FIG. 2B spatially related cropping to capture high-level features by optimizing their agreement. As depicted in FIG. 2A and the lower path 300 graphically illustrated in FIG. 3, embodiments leverage sub-volume order distortion to address learning perspectives (1) (i.e., intra-volume spatial relationships by sub-volume order prediction) and (2) (i.e., volume-wise fine-grained features by volume appearance recovery). For the acquisition of global features (3) (i.e., automatically capture high-level features by optimizing the agreement between features from two spatially related crops) at the patient level, embodiments employ spatially related cropping, as illustrated in FIG. 2B and the upper path 305 illustrated in FIG. 3.

The framework illustrated in FIG. 3 encompasses two learning perspectives: (1) acquiring high-level global features by optimizing the agreement between two spatially related views through the student-teacher learning paradigm (upper path 305), and (2) acquiring sub-volume relationships and fine-grained features through sub-volume order prediction and volume appearance recovery (lower path 300). Initially, sub-volume order distortion Fₚₑᵣₘ(·) (as depicted in FIG. 2A) is applied to an input patient volume. The model is trained to predict the original and correct sub-volume order, evaluated and corrected by Lₚₒₚ, depicted at 310, and to recover the volume's original appearance, evaluated and corrected by Lₐᵣ, as depicted at 315. Subsequently, spatially related cropping F_{src}(·) (as illustrated in FIG. 2B) is applied to the input patient volume. Two spatial-related views are sent to the student network 325 and teacher network 330, respectively. Additionally, L_{consist} is employed to assess and maximize the agreement between two global features of two views, with the teacher network remaining unaltered by this learning criterion, as depicted at 320. Instead, in both learning perspectives, the weight of teacher network is updated using the exponential moving average (EMA) based on the student's learning experience, as depicted at 335. These two perspectives are iteratively learned, contributing to the development of a robust, efficient, and accurate model. Finally, the teacher model is transferred to downstream tasks.

To elucidate the learning framework, certain mathematical notations are introduced in the following discussion.

### 3.1.Notations

Let S={S₁, S₂, S₃, . . . , S_{M}} be the collection of patient volumes, and Sᵢ∈R^{C×D×H×W}, where (D,H,W) is the resolution of the volume and C is the number of channels. The process then cyclically selects and applies one of the following functions: (a) volume order distortion Fperm(·) (the lower path 300 in FIG. 3), and (b) spatial-related cropping F_{src}(·) (the upper path 305 in FIG. 3).

Volume order distortion. This is illustrated intuitively in FIG. 2A, specifically, Sᵢ 200 is first divided into a sequence of non-overlapping sub-volumes Sᵢ^{sub}=(Sᵢ₁, Sᵢ₂, Sᵢ₃, . . . , S_{iK}), where K=D×H×W/p³ and (p, p, p) is the resolution of the sub-volume, as depicted at 205. Moreover, each sub-volume Sᵢₖ is paired with one unique 3D coordinate (z, x, y)ₖ, where 0≤z≤[D/p]−1, 0≤x≤[H/p]−1, 0≤y≤[w/p]−1, and zₖ, xₖ, yₖ ∈ Z, resulting in a sequence of sub-volume coordinates Ci=(Cᵢ₁, Cᵢ₂, Cᵢ₃, . . . , C_{iK}). Furthermore, both Sᵢ^{sub} and Cᵢ will be shuffled by the same random permutation operator, resulting in order re-arranged sub-volumes Sᵢ^{perm} (depicted at 210) and subvolume coordinates C^{perm}ᵢ. Based on Sᵢ^{perm} and its sequential order, a distorted volume Sᵢ^{dist} ∈R^{C×D×H×W} is re-constructed as depicted at 215. Sᵢ^{dist} is further processed by a Student transformer encoder g_{θs}^{enco}(·) (e.g., 3D Swin Transformer), such as depicted at 340, to obtain a set of contextual sub-volume feature maps Z^{perm}ᵢ=(Z^{perm}ᵢ₁,Z^{perm}ᵢ₂,Z^{perm}ᵢ₃, . . . ,Z^{perm}_{iK}). To perform sub-volume order prediction, Z^{perm}ᵢ is passed to a linear prediction head h_{θs}(·) to generate a set of predicted sub-volume orders P^{vo}ᵢ=h_{θs}(Z^{perm}ᵢ). Furthermore, Z^{perm}ᵢ is passed to a CNN-based decoder g_{θs}^{deco}(·), such as depicted at 350, and a volume P^{va}ᵢ=g_{θs}^{deco}(Z^{perm}ᵢ) is predicted for performing the appearance recovery task.

Spatial related cropping. As illustrated in FIG. 2B, each patient volume Sᵢ 200 is first up-sampled by 25% to Sᵢ^{interp}∈R^{C×D′×H′×W′} as depicted at 220. Sᵢ^{interp} is then divided into K′ number of non-overlapping sub-volumes, where K′=D′×H′×W′/p³. Two spatial-related crops Crop₁ (225), Crop₂ (230) ∈RC×D×H×W are generated by F_{src}(·), which ensures the number of overlapped sub-volumes between two crops falls within [(p−2)³, p³]. Both crops are concurrently input to Student and Teacher transformer encoder g_{θs}^{enco}(·) 340 and g_{θt}^{enco}(·) 345 to get the bottleneck local feature maps, s, t, respectively. An average pooling operator is performed on both local feature maps to generate unified dimension feature vectors sₚₒₒₗ, tₚₒₒₗ.

### 3.2. Sub-Volume Order Prediction and Volume Appearance Recovery

The goal of sub-volume order prediction is to predict the correct 3D coordinates of a sub-volume based on its appearance, and the volume appearance recovery aims to reconstruct the correct patient volume Sᵢ 200 given a distorted volume Sᵢ^{dist} 215. The expected prediction of P^{vo} and P^{va} are formulated as follows.

$${𝒫}_{i}^{vo} {=}^{!} {𝒞}_{i}^{perm} \qquad ( 1 )$$

$${𝒫}_{i}^{va} {=}^{!} {𝒮}_{i} \qquad ( 2 )$$

Following Schwichtenberg, “!=” in the above equations is defined to be “shall be (made) equal”. To better learn the spatial relationship between sub-volumes, the sub-volume order prediction is formulated as a regression task and the Student network trained by minimizing l₂ distance between the predicted sub-volume coordinates P^{vo}ᵢ=((z, x, y)^{pred}₁, (z, x, y)pred₂, . . . , (z, x, y)^{pred}_{K}) and the randomly shuffled coordinates C^{perm}ᵢ=((z, x, y)^{perm}₁, (z, x, y)perm₂, . . . , (z, x, y)^{perm}_{K}), the loss function of sub-volume order prediction being defined as:

$${ℒ}_{vop} = \frac{1}{B} {\sum }_{b = 1}^{B} {\sum }_{j = 1}^{K} {‖ {𝒫}_{ij}^{vo} - {𝒞}_{ij}^{perm} ‖}_{2}^{2} .$$

Moreover, the volume appearance recovery is formulated as a reconstruction task and the Student network trained by minimizing l₂ distance between the predicted volume P^{va}ᵢ and the original patient volume Sᵢ, wherein the appearance recovery loss is defined as:

$${ℒ}_{var} = \frac{1}{B} {\sum }_{b = 1}^{B} {‖ {𝒫}_{i}^{va} - {𝒮}_{i} ‖}_{2}^{2} ,$$

where B denotes the batch size in both loss functions. Both learning schemes are integrated with an overall loss function:

$${ℒ}_{vopar} = λ * {ℒ}_{vop} + ( 1 - λ ) * {ℒ}_{var} .$$

### 3.3. Global Embedding Consistency

Global embedding consistency aims to maximize the agreement between two crops' feature vectors sₚₒₒₗ, tₚₒₒₗ, generated by the Student and Teacher networks, respectively. A 3-layer MLP o_{θs}, o_{θt} is utilized to map two feature vectors into yₛ and yₜ, following which, a l₂-normalization is utilized such that:

$$\overline{{y}_{s}} = {y}_{s} / {‖ {y}_{s} ‖}_{2} and \overline{{y}_{t}} = {y}_{t} / {‖ {y}_{t} ‖}_{2} .$$

Finally, global embedding consistency loss is defined and the Student network trained by minimizing the l₂ distance between two normalized feature vectors.

$${ℒ}_{{θ}_{\text{?}} , {θ}_{t}}^{global} {=}^{Δ} {‖ \overline{{y}_{s}} - \overline{{y}_{t}} ‖}_{2}^{2} = 2 - 2 \cdot \frac{〈 {y}_{s} , {y}_{t} 〉}{{‖ {y}_{s} ‖}_{2} \cdot {‖ {y}_{t} ‖}_{2}}$$

$$\text{?} \text{indicates text missing or illegible when filed}$$

It should be noted that the Teacher's weight remains frozen and will be updated later based on the learning experience of the student.

### 3.4. Overall Training Scheme

The Student network is pretrained by cyclically propagating the loss Lᵥₒₚₐᵣ and L^{global}_{θs,θt}. Both encoder g_{θs}^{enco}(·) 340 and decoder g_{θs}^{deco}(·) 350 are updated by Lvopar, while L_{global}^{θs,θt} updates the encoder g_{θs}^{enco}(·) only. To further summarize and consolidate the knowledge learned by the two tasks, a Teacher model is introduced that shares the same architecture with the Student. The Teacher network is updated step-wise using exponential moving average (EMA) 335 based on the Student's learning experience. Eventually, the learned sub-volume-wise relationships, volume-wise fine-grained features, and overall context are refined in the Teacher model for future application-specific downstream tasks.

Thus, according to embodiments of the invention, a method performed by a system having at least a processor and a memory therein execute instructions for a self-supervised learning framework to learn fine-grained features, high-level global features, and contextual relationship features of anatomical structures in medical images of a plurality of patients, including the steps of receiving a plurality of computed tomography three-dimensional volumes for the plurality of patients (“patient volumes”), learning sub-volume relationships within the patient volumes through three dimensional sub-volume order prediction of correct positions in shuffled sub-volumes, learning fine-grained image features within the patient volumes through volume appearance recovery from a set of misplaced sub-volumes, and learning high-level global image features of anatomical structures in the patient volumes by maximizing an agreement between two spatially related views through a student-teacher network of the self-supervised learning framework.

According to embodiments, learning the sub-volume relationships within the patient volumes and learning the fine-grained image features within the patient volumes may be performed simultaneously using sub-volume order distortion.

According to embodiments, learning high-level global image features of anatomical structures in the patient volumes by maximizing an agreement between two spatially related views through a student-teacher network of the self-supervised learning framework may involve learning high-level global image features of anatomical structures in the patient volumes by maximizing an agreement between features of two spatially related cropped views of the patient volumes.

Further, according to embodiments of the invention, a method performed by a system having at least a processor and a memory therein to execute instructions for a self-supervised learning (SSL) model learns fine-grained features, high-level global features, and contextual relationship features of anatomical structures in medical images of a plurality of patients. The process may involve receiving a plurality of computed tomography three-dimensional volumes for the plurality of patients (“patient volumes”), applying a sub-volume order distortion to the patient volumes to create a distorted patient volume, training the SSL model to predict an original and a correct sub-volume order of each patient volume, recovering an original appearance of each patient volume, applying spatially related cropping to each patient volume, comprising transmitting two spatial-related views to a student and teacher network, respectively, of the SSL model, and assessing and maximizing an agreement between two global features of the two spatially-related views.

According to embodiments, assessing and maximizing the agreement between two global features of the two spatially-related views may involve assessing and maximizing the agreement between two global features of the two spatially-related views with the teacher network remaining unaltered.

Further, according to embodiments, assessing and maximizing an agreement between two global features of the two spatially-related views may involve updating a weight of the teacher network using an exponential moving average (EMA) 335 based on the student network.

According to embodiments, applying a sub-volume order distortion to the patient volumes may involve dividing the patient volume into a sequence of non-overlapping sub-volumes, pairing each sub-volume in the sequence with a unique three-dimensional coordinate, resulting in a sequence of sub-volume coordinates, shuffling, via a random permutation operator, the sequence of sub-volumes and the sequence of sub-volume coordinates, resulting in re-arranged sub-volumes and sub-volume coordinates, re-constructing a distorted patient volume based on the re-arranged sub-volumes and their sequential order, processing the distorted patient volume via a student transformer encoder to obtain a set of contextual sub-volume feature maps, passing the set of contextual sub-volume feature maps to a linear prediction head, generating via the linear prediction head a set of predicted sub-volume orders, passing the set of contextual sub-volume feature maps to a CNN-based decoder, and predicting, via the CNN-based decoder, a volume for performing an appearance recovery task.

According to an embodiment, the step of applying spatially related cropping to each patient volume involves up-sampling each patient volume, resulting in up-sampled patient volumes, dividing each up-sampler patient volume into a plurality of non-overlapping sub-volumes, generating two spatially-related crops from the plurality of non-overlapping sub-volumes, concurrent, transferring the two spatially-related crops to a student and a teacher transformer encoder, generating, via the student and teacher transformer encoders, two local feature maps, and performing an average pooling operator on the two local feature maps to generate unified dimension feature vectors.

In some embodiments, recovering the original appearance of each patient volume comprises reconstructing the patient volume given the distorted patient volume.

### 4. Experiments

### 4.1.1. Pretraining Setup

ASA is pretrained on a collection of three datasets, LUNA16, TICA Colon, and HNSCC, containing various body regions such as the chest, head, neck, abdomen, and pelvis. Embodiments follow the data split for a fair comparison. All the patient volumes are re-sampled into the same voxel space, (2.0, 1.5, 1.5) for z, x, and y dimensions respectively. The CT volume intensities are then clipped from −175 to 250 and the clipped intensity values normalized between 0 to 1. For the sub-volume order prediction and volume appearance recovery tasks, the volume is directly resized into Sᵢ∈R^{1×128×128×128} and the sub-volume size is set to be S^{sub}ᵢₖ∈R^{1×16×16×16, resulting in K=}512 non-overlapping shuffle-able sub-volumes. After mapping the sub-volume indices into a 3D coordination system, 512 distinct coordinates are obtained, where 0≤z≤7, 0 ≤x≤7, 0≤y≤7, and z, x, y ∈Z. To stabilize the order prediction training, the coordinates are normalized between −3 to 3. For the global embedding consistency training task, after normalizing the voxel space and intensity, the original volume is up-sampled to Sᵢ^{interp}∈R^{1×160×160×160}, from which two spatially related crops Crop₁, Crop₂∈R^{1×128×128×128} are obtained. The Swin UNETR architecture is utilized as the student and teacher networks, where the teacher network is updated by the student network step-wise via EMA with an updating parameter of 0.9. Both networks are pretrained with SGD optimizer with a learning rate of le⁻² for 150 epochs on four NVIDIA A100 GPUs. For SimMIM base line pretraining, the official implementation is followed and the method re-implemented in 3D on Swin UNETR. A 50% masking ratio is applied, and each mask has a size of 16×16×16. All pretraining frameworks are implemented on PyTorch with MONAI for data preprocessing.

### 4.2. Fine-Tuning Setup

The ASA model is fine-tuned according to some embodiments as well as baseline models on diverse abdomen organ segmentation tasks. In the preprocessing step for all tasks, all scans are first re-sampled to a uniform voxel space, (1.5 (2.0 for BTCV), 1.5, 1.5) for z, x, and y dimensions. Subsequently, the intensity values are clipped within the range of −175 to 250 and then normalized to a scale between 0 and 1. Moreover, during training, 128×128×128 voxels are randomly sampled, incorporating spatial padding if any dimension is smaller than the specified input size. Data augmentation techniques, including random flips, rotations, and intensity shifts, are employed during training with probabilities of 0.1, 0.1, and 0.5, respectively. All tasks are fine-tuned utilizing the AdamW optimizer with a learning rate of le⁻⁴. The training is performed on the Dice similarity coefficient loss for 30,000 iterations, employing a batch size of 1. The implementation of all downstream tasks is carried out using PyTorch and MONAI and is run on a single NVIDIA A100 GPU. In each experiment, five independent runs were performed and the average Dice score presented as the metric for evaluating the experiment results.

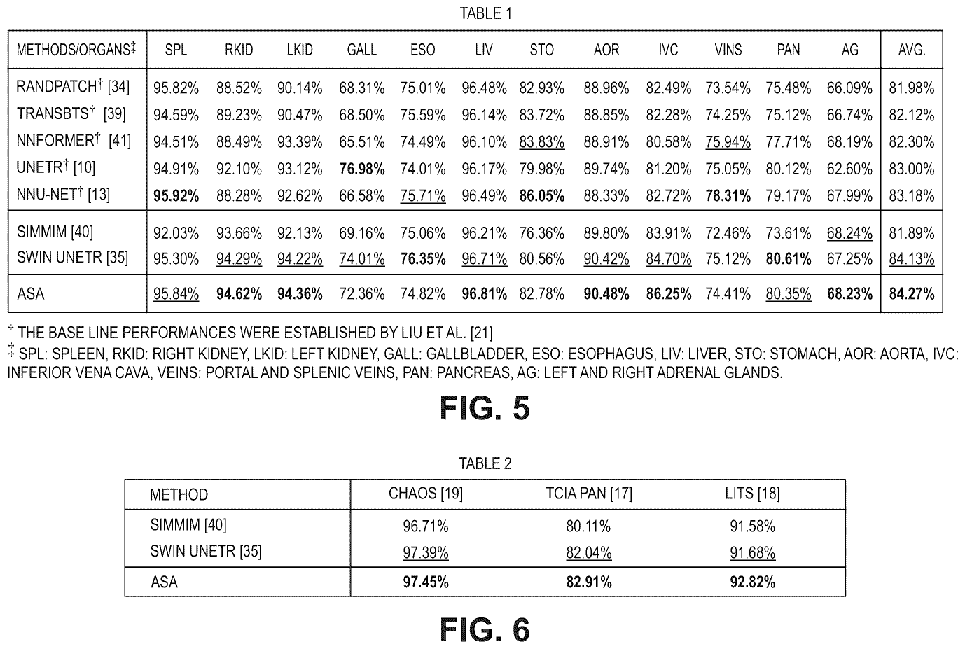

BTCV: The Beyond the Cranial Vault (BTCV) dataset comprises CT scans from 30 patients, with each scan accompanied by 14 manual segmentation annotations. These annotations consist of one background and 13 different organs. Following the approach in Ali Hatamizadeh, Vishwesh Nath, Yucheng Tang, Dong Yang, Holger R Roth, and Daguang Xu, “Swin UNETR: Swin Transformers for Semantic Segmentation of Brain Tumors in MRI Images,” International MICCAI Brainlesion Workshop, 636 pages 272-284, Springer, 2021 and Yucheng Tang, Dong Yang, Wenqi Li, Holger R Roth, Bennett Landman, Daguang Xu, Vishwesh Nath, and Ali Hatamizadeh, “Self-Supervised Pre-Training of Swin Transformers for 3D Medical Image Analysis,” Proceedings of the IEEE/CVF Conference on Computer Vision and Pattern Recognition, pages 20730-20740, 2022, a split of 24 samples is established for training and six samples for testing. A 14-class segmentation task was formulated, encompassing segmenting background, spleen, right kidney, left kidney, gallbladder, esophagus, liver, stomach, aorta, inferior vena cava, portal vein, splenic vein, pancreas, right adrenal gland, and left adrenal gland. Table 1 in FIG. 5 presents the performance metrics for organ segmentations, combining the results for the right and left adrenal glands to facilitate comparison with reported performances in other works. The experiment results on the BTCV validation dataset revealed that the novel approach described herein surpasses both fully-supervised and self-supervised techniques in achieving the highest average Dice score across all organs. More specifically, the novel method excels in segmenting 7 out of 12 organs, outperforming all fully supervised learning methods, and surpasses state-of-the-art self-supervised learning techniques in segmenting 8 out of 12 organs. This underscores the superior knowledge demonstrated by the model. The best performances are in bold and the second best are underlined in Table 1.

CHAOS: The Combined Healthy Abdominal Organ Segmentation (CHAOS) dataset is composed of CT scans from 20 patients, each accompanied by five segmentation annotations. These annotations include one background and four various organs, but the focus in this task is solely on liver segmentation. In accordance with Jie Liu, Yixiao Zhang, Jie-Neng Chen, Junfei Xiao, Yongyi Lu, Bennett A Landman, Yixuan Yuan, Alan Yuille, Yucheng Tang, and Zongwei Zhou, “CLIP-Driven Universal Model for Organ Segmentation and Tumor Detection,” Proceedings of the IEEE/CVF International Conference on Computer Vision, pages 21152-21164, 2023, a split of 16 samples for training and 4 samples for testing is established. A 2-class segmentation task is designed, encompassing the background and liver classes. Table 2, presented in FIG. 6, details the performance metrics for liver segmentation. The results of the experiment on single-organ segmentation tasks demonstrate that embodiments of the invention outperform both state-of-the-art self-supervised learning methods. This highlights the novel method's ability to effectively capture global features, particularly on larger objects. The best performances are in bold and the second best are underlined.

Pancreas-CT: The Pancreas-CT dataset comprises 80 abdominal contrast-enhanced 3D CT scans from 53 male and 27 female subjects, each scan paired with two manual segmentation annotations for background and pancreas. Following the protocol outlined in Liu, et. al., a split of 64 samples for training and 16 samples for testing is established. A 2-class segmentation task is formulated, including the background and pancreas classes. Table 2 presents the performance metrics for pancreas segmentation.

LiTS: The Liver Tumor Segmentation Benchmark (LiTS) comprises 130 CT scans, each paired with three manual segmentation annotations. These annotations include one for the background, one for the liver organ, and one for the liver tumor. Following the methodology outlined in Liu et al., a split of 94 samples for training and 36 samples for testing is established. The segmentation task is structured as a three-class problem, encompassing the background, liver, and liver tumor. Performance metrics for the liver segmentation are presented in Table 2, presented in FIG. 6.

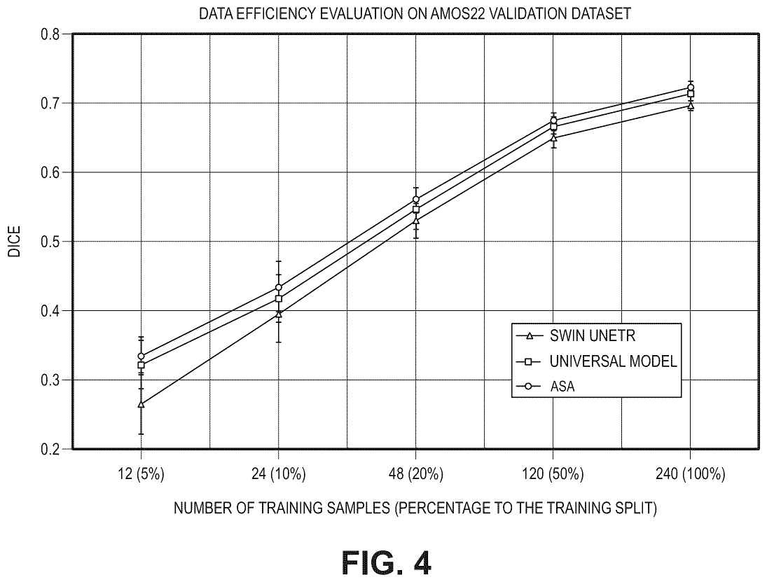

AMOS2022: The Multi-Modality Abdominal Multi-Organ Segmentation Challenge (AMOS2022) consists of 360 CT scans, each scan paired with voxel-level annotations for 15 abdominal organs and one background class. Adhering to the official training split of 240 samples and 120 samples for testing, a 16-class segmentation task is formulated. This task includes segmenting the background, spleen, right kidney, left kidney, gall bladder, esophagus, liver, stomach, aorta, postcava, pancreas, right adrenal gland, left adrenal gland, duodenum, bladder, and prostate/uterus. FIG. 4 graphically illustrates data efficiency evaluation conducted on ASA and base-line methods, depicting the mean Dice scores for all organs across various finetuning data ratios. In particular, FIG. 4 assesses the data efficiency of the described embodiments, Swin UNETR, and the Universal Model on the AMOS22 validation dataset. In the context of few sample fine-tuning, ASA model showcases its efficacy when compared to both baseline models, underscoring the robust features inherent in our model.

The effectiveness of the embodiments was validated using the AMOS2022 dataset, conducting a comparative analysis that contrast the ASA model against two state-of-the art (SoTA) counterparts: Swin UNETR, a self-supervised learning method designed for 3D medical segmentation tasks, and the Universal Model, a leading image-text supervised learning approach.

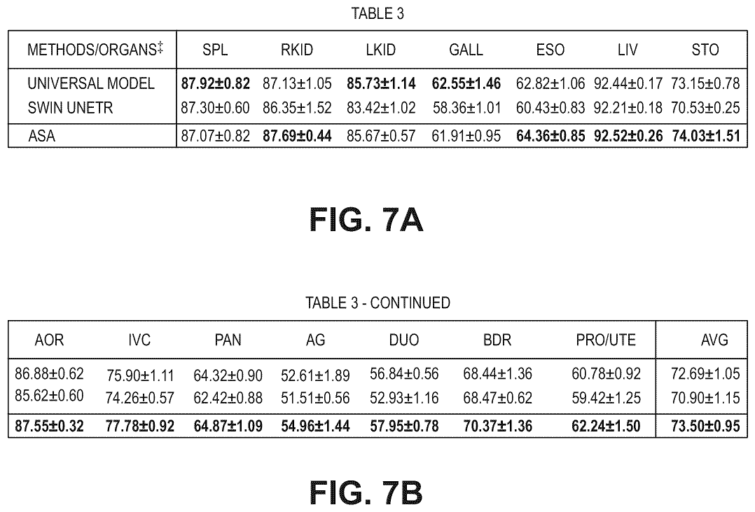

It is worth noting that the Universal Model has undergone training exposure to a segment of the training split within the AMOS22 dataset. In contrast, both the ASA model and the Swin UNETR model have been exposed to any portion of the AMOS22 dataset during their pretraining phases. This divergence in training data exposure is crucial for assessing the generalization capabilities and adaptability of each model, particularly in the context of unseen data, a scenario mirroring real-world applications. To conduct a fair evaluation, all models are finetuned on the AMOS2022 training split and subsequently test their performance on the validation split. As depicted in Table 3 in FIGS. 7A and 7B, remarkably, the ASA model emerges as the outstanding performer, outperforming both the Swin UNETR and Universal Model baseline approaches. The superiority of the ASA model is highlighted by its achievement of the highest average Dice score across all 14 organs. Specifically, the ASA model not only secures the top position in segmenting 11 out of the 14 organs but also consistently exhibits the second-best performance for the remaining three organs. This nuanced and detailed analysis underscores the robustness and efficacy demonstrated by the ASA model across a spectrum of organ segmentation tasks. In Table 3, the outcomes of the experiments on the AMOS22 validation dataset underscore the superior performance of the ASA model, surpassing both supervised and self-supervised state-of-the-art baseline models. Notably, the ASA model achieves the highest average Dice score across all 14 organs, positioning it as a standout performer. In a detailed breakdown, the ASA model secures the top position in segmenting 11 out of the 14 organs, while for the remaining three, it consistently achieves the second-best performance. This comprehensive dominance in organ segmentation highlights the remarkable effectiveness demonstrated by the model. The best performances are in bold and the second best are underlined. Statistical analysis is conducted for each organ by comparing the best with the other baseline models. The-highlighted boxes indicate no statistically significant difference at level p=0.05.

Swin UNETR: Swin UNETR undergoes a pretraining phase involving three common self-supervised learning tasks on five publicly accessible CT datasets (superset of ASA), encompassing TCIA Covid19, LiDCH, NSCC, LUNA16, and TICA Colon, comprising a total of 5,050 subjects. To fine-tune the pretrained weights, the pretrained model is obtained from its official GitHub release. Given the availability of only encoder weights, the decoder part randomly initialized for all subsequent evaluations in downstream tasks.

### 5. Results

The model according to embodiments undergoes a thorough comparison with both fully-supervised and self-supervised baselines, revealing its superior performance across various metrics related to multi-organ segmentation task (Table 1-FIG. 5), single organ segmentation tasks (Table 2-FIG. 6), and data efficiency examination task (FIG. 4).

### 5.1. Multi-Organ Segmentation Challenge on BTCV

The ASA model and baseline models are fine-tuned according to some embodiments end-to-end on the BTCV training split and a comprehensive comparison of the model is compared against five fully-supervised and two self-supervised learning baselines. As depicted in Table 1 (FIG. 5) the ASA model excels beyond all reported methods on thirteen organ segmentation tasks (left and right adrenal glands combined) on the BTCV validation set. Particularly noteworthy is its superior performance over all five fully-supervised learning methods in segmenting seven out of twelve organs. This underscores the effectiveness of the method, which is pretrained on three chest and abdomen datasets, in acquiring more generic appearance features for a variety of abdomen organs. Additionally, the model surpasses the self-supervised pretraining methods SimMIM and Swin UNETR, recognized as state-of-the-art (SoTA) in 2D natural/medical imaging and 3D medical imaging domains, respectively. The substantial margin by which the method according to embodiments outperforms SimMIM underscores the efficacy of learning anatomical relationships. Furthermore, it is noteworthy that the model outperforms Swin UNETR which is pretrained via three proxy tasks to learn volume-level discriminative and rotation-invariant features for the thoracic and abdominal regions using five datasets. This observation implies that more robust features can be learned by capturing anatomical structure via sub-volume order prediction and depicting fine-grained appearance features results via volume appearance recovery.

### 5.2. Single Organ Segmentation on Three Datasets

To assess the model's adaptability to specific organs, all models are fine-tuned on a series of single-organ segmentation tasks, including liver (CHAOS, LiTS) and pancreas (TCIA Pan). As depicted in Table 2 (FIG. 6), the ASA model surpasses both baseline self-supervised learning methods. As the liver is a sizable organ in the abdominal region, all models demonstrate high performances as measured by the Dice score. Notably, the method according to embodiments attains the highest score, underscoring its superiority in delineating intricate edge details. In pancreas segmentation, the method outperforms SimMIM by a significant margin and surpasses Swin UNETR by a more modest margin, highlighting the enhanced adaptability of features acquired through the method, which effectively captures spatial relationships and fine-grained features.

### 5.3 Data Efficiency Evaluation on AMOS22

ASA underscores its superiority by consistently outperforming Swin UNETR, a SoTA self-supervised learning method on 3D medical segmentation task benchmarks and Universal Model, a SoTA image-text supervised learning method in small data regimes. This emphasizes the superiority of ASA model, providing richer information that can be utilized more efficiently. All three models may be fine-tuned on subsets comprising 12 (5%), 24 (10%), 48 (20%), 120 (50%), and 240 (100%) randomly selected samples from the official training split of AMOS22. To ensure fairness across diverse random samples, five independent runs were conducted and their average performances reported. The mean Dice scores of 15 organ segmentation performances in this task are also reported. As illustrated in FIG. 4, even with 12 (5%) training samples, the ASA model outperforms Swin UNETR significantly. While the supervised Universal Model exhibits superior performance compared to Swin UNETR, it still falls slightly below the performance of the method according to embodiments. Despite improvements in performance with the increase in training samples for all methods, the method consistently outperforms both of the other methods across all data regimes. This underscores the effectiveness of the method according to embodiments in extracting fine-grained features and organ appearance information, even when pretrained on fewer datasets. A full performance report is provided above in the discussion of Table 3 provided in FIGS. 7A and 7B.

The spatial relationships of local embeddings generated by ASA, Universal Model, and Swin UNETR models were explored on the AMOS22 testing dataset, comprising 240 unseen samples. Employing the preprocess strategies described in Section 4.2, each 128×128×128 input volume is divided into 512 (8×8×8) sub-volumes, each sized 16×16×16. Spatially related sub-cubes at central indices 3-3-3, 4-4-4, and 5-5-5, as well as diagonal sub-volumes at indices 0-0-0 and 7-7-7, are selected for examination. Across all test samples, embeddings are generated for these sub-volumes by the three models.

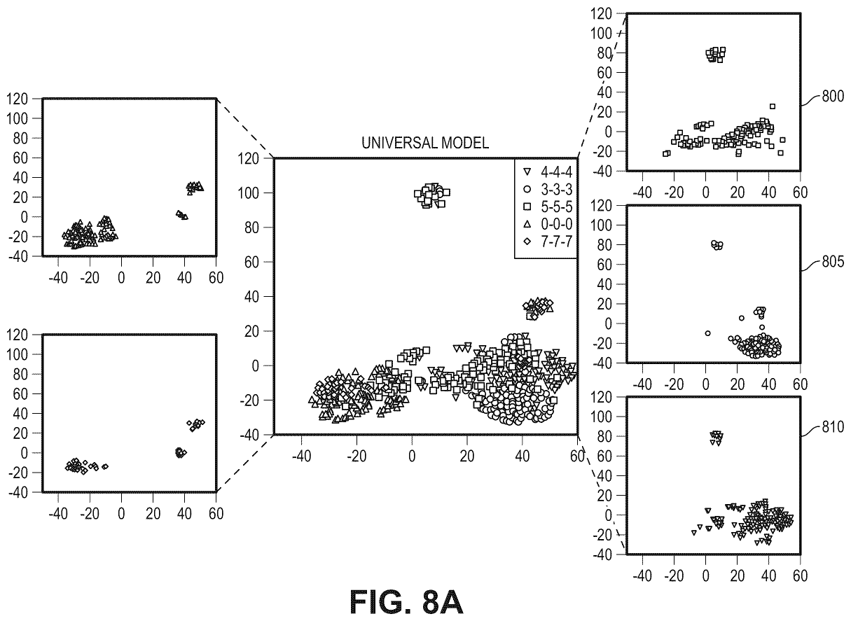

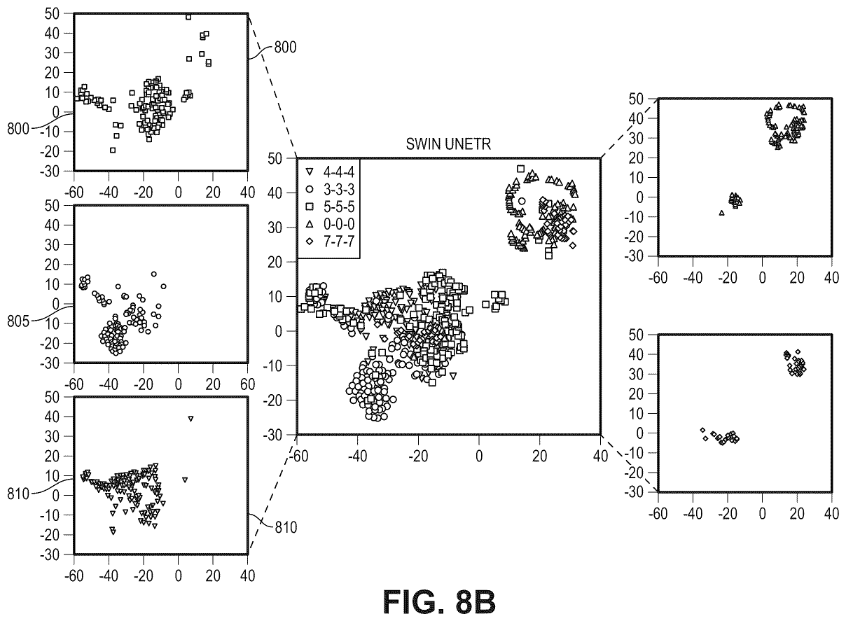

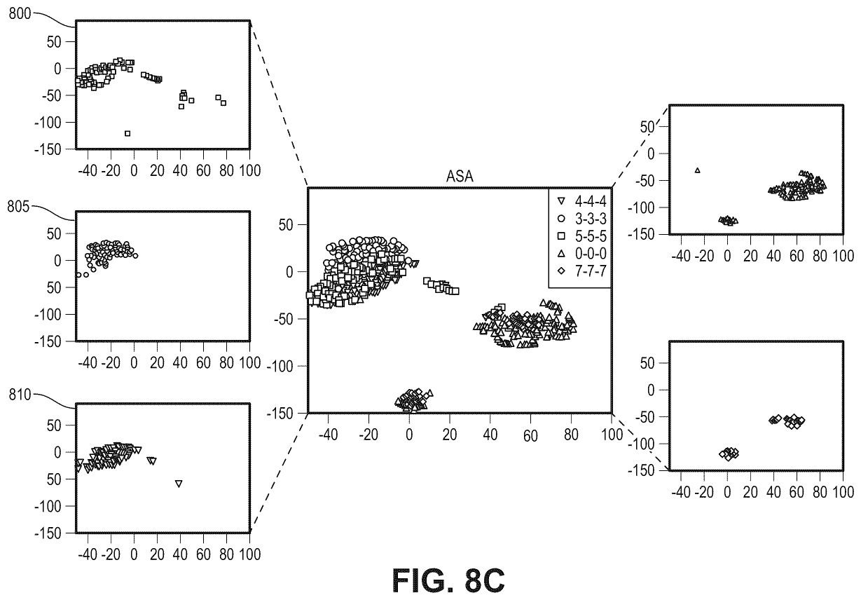

FIGS. 8A, 8B and 8C employ circles to depict spatially related embeddings and cross signs for the diagonal embeddings. Despite a few outliers, the ASA model demonstrates strong relational properties among the spatially related sub-volumes, where circles 800, circles 805, and circles 810 are closely clustered. In contrast, the spatially related sub-volume embeddings produced by the Universal Model and Swin UNETR exhibit a more dispersed distribution, indicating weaker spatial relationships. Interestingly, for diagonal sub-volumes, all models exhibit similar performance, likely due to these sub-volumes containing only 0 intensity values, making them easily clustered based on appearance features.

In summary, the ASA model excels in preserving spatial relationships among related sub-volumes, as evidenced by tight clustering in the visual representation. Conversely, the Universal Model and Swin UNETR exhibit less cohesive spatial relationships in their embeddings, with a more scattered distribution.

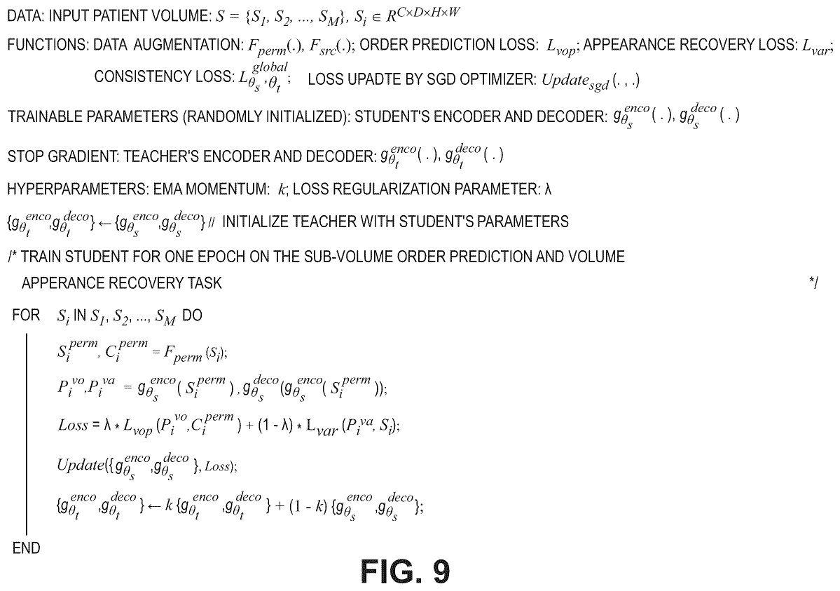

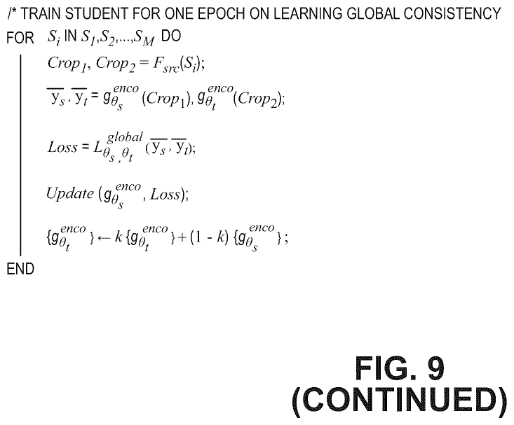

ASA adopts a teacher-student model architecture, with the student being an active learner responsible for acquiring knowledge pertaining to both sub-volume relationships and volume appearance information. Furthermore, a collaborative effort is established between the student and teacher to enhance the agreement of their embeddings derived from spatially related views extracted from the volume. The overall learning process is outlined in the algorithm presented in FIG. 9, where ASA employs a cyclic pretraining approach to formulate learning perspectives.

The algorithm in FIG. 9 describes one round of pretraining initiated by ASA. Initially, ASA guides the student in learning sub-volume relationships and volume appearance from the entirety of the training samples. Subsequently, the student is prompted to enhance its understanding of global information by maximizing the embedding agreement with the teacher through two spatially related crops. These two tasks are iteratively applied to the student, and at the end of each task, the accumulated knowledge is incorporated into the teacher network through exponential moving average (EMA). This iterative process results in both the teacher and student networks acquiring a comprehensive understanding of solid anatomical patterns, fine-grained appearance information, and global anatomy features.

The teacher network, enriched and consolidated through the student's learning experiences, is then reused and transferred to task-specific targets within applications. This transfer of knowledge ensures that the teacher network, representing a distilled and refined form of accumulated expertise, contributes to the effectiveness of ASA in application-specific tasks.

### 6. Ablation Study

### 6.1. Comparison Among Different Learning Tasks

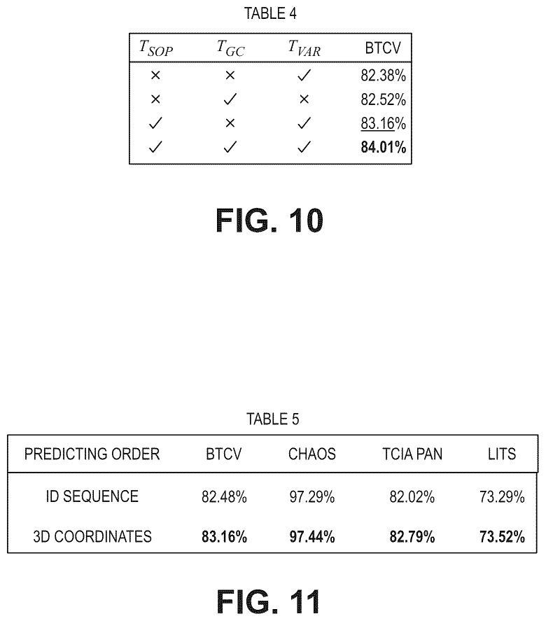

An ablation study was conducted to demonstrate the effectiveness of individual training tasks and the combination of multiple tasks on BTCV segmentation task. As depicted in Table 4, the model trained by volume appearance recovery or global consistency tasks that yields comparable performance. However, leveraging the combined benefits of sub-volume order prediction and volume appearance recovery tasks enhances performance, attributed to the model's capacity to learn sub-volume relationships and extract fine-grained features. Lastly, as indicated in the final row of Table 4 provided in FIG. 10, incorporating consistency to maximize the agreement between two views from the same patient volume elevates the performance of the BTCV downstream task even further.

### 6.2. Predicting 1D Sub-Volume Sequences and 3D Sub-Volume Coordinates

The efficacy of employing 1D sub-volume order presentation (e.g., 1, 2, 3, . . . , k), and 3D sub-volume order presentation (e.g., (0, 0, 0), . . . , (3, 3, 5), . . . , (z, x, y)), was assessed. The results presented in Table 5 in FIG. 11 indicate that when making predictions based on 3D sub-volume order presentation, performance across all downstream tasks consistently surpasses that achieved with 1D sub-volume order prediction. This observation underscores the importance of utilizing 3D sub-volume order presentation for enhanced model performance.

### 7. Conclusion

The described embodiments provide a novel self-supervised learning method referred to herein as ASA, capitalizing on the unique attributes of medical images to acquire robust global features, intra-volume relationships, and detailed appearance features. Furthermore, ASA introduces at least a novel pretraining paradigm, employing a student-teacher network to cyclically attain diverse learning perspectives. Thoroughly examined through extensive experiments, ASA has proven its effectiveness and efficiency.

Embodiments of the invention contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via a public Internet.

The system may further include peripheral device (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A method performed by a system having at least a processor and a memory therein to execute instructions for a self-supervised learning framework to learn fine-grained features, high-level global features, and contextual relationship features of anatomical structures in medical images of a plurality of patients, comprising:
receiving a plurality of computed tomography three-dimensional volumes for the plurality of patients (“patient volumes”);
learning sub-volume relationships within the patient volumes through three-dimensional sub-volume order prediction of correct positions in shuffled sub-volumes;
learning fine-grained image features within the patient volumes through volume appearance recovery from a set of misplaced sub-volumes; and
learning high-level global image features of anatomical structures in the patient volumes by maximizing an agreement between two spatially related views through a student-teacher network of the self-supervised learning framework.

2. The method of claim 1, wherein learning the sub-volume relationships within the patient volumes and learning the fine-grained image features within the patient volumes are performed simultaneously using sub-volume order distortion.

3. The method of claim 1, wherein learning high-level global image features of anatomical structures in the patient volumes by maximizing the agreement between two spatially related views through the student-teacher network of the self-supervised learning framework comprises learning high-level global image features of anatomical structures in the patient volumes by maximizing an agreement between features of two spatially related cropped views of the patient volumes.

4. A method performed by a system having at least a processor and a memory therein to execute instructions for a self-supervised learning (SSL) model to learn fine-grained features, high-level global features, and contextual relationship features of anatomical structures in medical images of a plurality of patients, comprising:
receiving a plurality of computed tomography three-dimensional volumes for the plurality of patients (“patient volumes”);
applying a sub-volume order distortion to each of the patient volumes to create a corresponding distorted patient volume;
training the SSL model to predict an original and a correct sub-volume order of each patient volume;
recovering an original appearance of each patient volume;
applying spatially related cropping to each patient volume, comprising transmitting two spatial-related views to a student and teacher network, respectively, of the SSL model; and
assessing and maximizing an agreement between two global features of the two spatially-related views.

5. The method of claim 4, wherein assessing and maximizing the agreement between two global features of the two spatially-related views comprises assessing and maximizing the agreement between two global features of the two spatially-related views with the teacher network remaining unaltered.

6. The method of claim 4, wherein assessing and maximizing an agreement between two global features of the two spatially-related views comprises updating a weight of the teacher network using an exponential moving average (EMA) based on the student network.

7. The method of claim 4, wherein applying a sub-volume order distortion to the patient volumes comprises:
dividing the patient volume into a sequence of non-overlapping sub-volumes;
pairing each sub-volume in the sequence with a unique three-dimensional coordinate, resulting in a sequence of sub-volume coordinates;
shuffling, via a random permutation operator, the sequence of sub-volumes and the sequence of sub-volume coordinates, resulting in re-arranged sub-volumes and sub-volume coordinates;
re-constructing a distorted patient volume based on the re-arranged sub-volumes and their sequential order;
processing the distorted patient volume via a student transformer encoder to obtain a set of contextual sub-volume feature maps;
passing the set of contextual sub-volume feature maps to a linear prediction head;
generating via the linear prediction head a set of predicted sub-volume orders;
passing the set of contextual sub-volume feature maps to a CNN-based decoder; and
predicting, via the CNN-based decoder, a volume for performing an appearance recovery task.

8. The method of claim 7, wherein applying spatially related cropping to each patient volume comprises:
up-sampling each patient volume, resulting in up-sampled patient volumes;
dividing each up-sampler patient volume into a plurality of non-overlapping sub-volumes;
generating two spatially-related crops from the plurality of non-overlapping sub-volumes;
concurrently transferring the two spatially-related crops to a student and a teacher transformer encoder;
generating, via the student and teacher transformer encoders, two local feature maps; and
performing an average pooling operator on the two local feature maps to generate unified dimension feature vectors.

9. The method of claim 4 wherein recovering the original appearance of each patient volume comprises reconstructing the patient volume given the distorted patient volume.

10. A system comprising:
a memory to store instructions;
a processor to execute the instructions stored in the memory;
a receive interface to receive computed tomography three-dimensional volumes for a plurality of patients (“patient volumes”);
wherein the system is configured to perform a self-supervised learning (SSL) model to learn fine-grained features, high-level global features, and contextual relationship features of anatomical structures in medical images of a plurality of patients, by executing the instructions via the processor for:
applying a sub-volume order distortion to the patient volumes to create a distorted patient volume;
training the SSL model to predict an original and a correct sub-volume order of each patient volume;
recovering an original appearance of each patient volume;
applying spatially related cropping to each patient volume, comprising transmitting two spatial-related views to a student and teacher network, respectively, of the SSL model; and
assessing and maximizing an agreement between two global features of the two spatially-related views.

11. The system of claim 10, wherein assessing and maximizing the agreement between two global features of the two spatially-related views comprises assessing and maximizing the agreement between two global features of the two spatially-related views with the teacher network remaining unaltered.

12. The system of claim 10, wherein assessing and maximizing an agreement between two global features of the two spatially-related views comprises updating a weight of the teacher network using an exponential moving average (EMA) based on the student network.

13. The system of claim 10, wherein applying a sub-volume order distortion to the patient volumes comprises:
dividing the patient volume into a sequence of non-overlapping sub-volumes;
pairing each sub-volume in the sequence with a unique three-dimensional coordinate, resulting in a sequence of sub-volume coordinates;
shuffling, via a random permutation operator, the sequence of sub-volumes and the sequence of sub-volume coordinates, resulting in re-arranged sub-volumes and sub-volume coordinates;
re-constructing a distorted patient volume based on the re-arranged sub-volumes and their sequential order;
processing the distorted patient volume via a student transformer encoder to obtain a set of contextual sub-volume feature maps;
passing the set of contextual sub-volume feature maps to a linear prediction head;
generating via the linear prediction head a set of predicted sub-volume orders;
passing the set of contextual sub-volume feature maps to a CNN-based decoder; and
predicting, via the CNN-based decoder, a volume for performing an appearance recovery task.

14. The system of claim 13, wherein applying spatially related cropping to each patient volume comprises:
up-sampling each patient volume, resulting in up-sampled patient volumes;
dividing each up-sampler patient volume into a plurality of non-overlapping sub-volumes;
generating two spatially-related crops from the plurality of non-overlapping sub-volumes;
concurrently transferring the two spatially-related crops to a student and a teacher transformer encoder;
generating, via the student and teacher transformer encoders, two local feature maps; and
performing an average pooling operator on the two local feature maps to generate unified dimension feature vectors.

15. The system of claim 10 wherein recovering the original appearance of each patient volume comprises reconstructing the patient volume given the distorted patient volume.

16. A non-transitory computer-readable storage media having instructions stored thereupon that, when executed by a system having at least a processor and a memory therein, perform a self-supervised learning (SSL) model to learn fine-grained features, high-level global features, and contextual relationship features of anatomical structures in medical images of a plurality of patients, by executing the instructions via the processor comprising:
receiving computed tomography three-dimensional volumes for a plurality of patients (“patient volumes”);
applying a sub-volume order distortion to the patient volumes to create a distorted patient volume;
training the SSL model to predict an original and a correct sub-volume order of each patient volume;
recovering an original appearance of each patient volume;
applying spatially related cropping to each patient volume, comprising transmitting two spatial-related views to a student and teacher network, respectively, of the SSL model; and
assessing and maximizing an agreement between two global features of the two spatially-related views.

17. The non-transitory computer-readable storage media of claim 16, wherein assessing and maximizing the agreement between two global features of the two spatially-related views comprises assessing and maximizing the agreement between two global features of the two spatially-related views with the teacher network remaining unaltered.

18. The non-transitory computer-readable storage media of claim 16, wherein assessing and maximizing an agreement between two global features of the two spatially-related views comprises updating a weight of the teacher network using an exponential moving average (EMA) based on the student network.

19. The non-transitory computer-readable storage media of claim 16, wherein applying a sub-volume order distortion to the patient volumes comprises:
dividing the patient volume into a sequence of non-overlapping sub-volumes;
pairing each sub-volume in the sequence with a unique three-dimensional coordinate, resulting in a sequence of sub-volume coordinates;
shuffling, via a random permutation operator, the sequence of sub-volumes and the sequence of sub-volume coordinates, resulting in re-arranged sub-volumes and sub-volume coordinates;
re-constructing a distorted patient volume based on the re-arranged sub-volumes and their sequential order;
processing the distorted patient volume via a student transformer encoder to obtain a set of contextual sub-volume feature maps;
passing the set of contextual sub-volume feature maps to a linear prediction head;
generating via the linear prediction head a set of predicted sub-volume orders;
passing the set of contextual sub-volume feature maps to a CNN-based decoder; and
predicting, via the CNN-based decoder, a volume for performing an appearance recovery task.

20. The non-transitory computer-readable storage media of claim 19, wherein applying spatially related cropping to each patient volume comprises:
up-sampling each patient volume, resulting in up-sampled patient volumes;
dividing each up-sampler patient volume into a plurality of non-overlapping sub-volumes;
generating two spatially-related crops from the plurality of non-overlapping sub-volumes;
concurrently transferring the two spatially-related crops to a student and a teacher transformer encoder;
generating, via the student and teacher transformer encoders, two local feature maps; and
performing an average pooling operator on the two local feature maps to generate unified dimension feature vectors.
